(19) United States
(12) Patent Application Publication
(10) Pub. No.: US 2012/0004517 A1
(43) Pub. Date: Jan. 5, 2012

(54) BLOOD PRESSURE MONITOR AND PULSE OXIMETER SYSTEM FOR ANIMAL RESEARCH

(75) Inventors: Eric W. Starr, Allison Park, PA (US); Bernard F. Hete, Kittanning, PA (US)

(73) Assignee: STARR LIFE SCIENCES CORP., Oakmont, PA (US)

(21) Appl. No.: 12/980,324

(22) Filed: Dec. 28, 2010

Related U.S. Application Data
(63) Continuation-in-part of application No. 12/249,044, filed on Oct. 10, 2008, now Pat. No. 7,857,768.
Application No. 12/249,044 is a continuation-in-part of application No. 12/330,501, filed on Dec. 8, 2008.
(60) Provisional application No. 60/978,813, filed on Oct. 10, 2007.
(60) Provisional application No. 61/108,010, filed on Oct. 23, 2008.
(60) Provisional application No. 60/992,880, filed on Dec. 6, 2007.

(51) Int. Cl. A61B 5/02; A61B 5/1455; A61B 5/022

(52) U.S. Cl. 600/301; 600/499

(57) ABSTRACT

An integrated blood pressure monitor and pulse oximeter system includes a blood flow occlusion member configured to selectively occlude blood flow through an appendage of the animal (e.g., the neck or tail); a sensor coupled to the blood flow occlusion member detecting a degree of operation thereof; Light sources coupled to the tail closer to the distal end of the tail than the tail blood flow occlusion member, and selectively directing light of two different wavelengths into the appendage; a light receiver coupled to the appendage and selectively receiving a signal associated with light directed into the appendage from the light sources; and a controller configured to selectively determine blood pressure parameters from the data and pulse oximeter parameters from the data.

### RELATED APPLICATIONS

This application is a Continuation-in-part of U.S. patent application Ser. No. 12/249,044, entitled “Integrated Tail Mounted Blood Pressure Monitor and Pulse Oximeter System for Animal Research” now U.S. Pat. No. 7,857,768. U.S. patent application Ser. No. 12/249,044 claims the benefit of U.S. Provisional Patent Application Ser. No. 60/978,813, filed Oct. 10, 2007 entitled “Integrated Tail Mounted Blood Pressure Monitor and Pulse Oximeter System for Animal Research”

This application is a continuation in part of U.S. patent application Ser. No. 12/330,501, entitled “Noninvasive Photoplethysmographic Sensor Platform for Mobile Animals” which published as U.S. Patent Publication Serial Number 2009-0149727 on Jun. 11, 2009. U.S. patent application Ser. No. 12/330,501 claims the benefit of U.S. Provisional Patent Application Ser. No. 61/108,010 entitled “Neck Collar Clip Small Animal Pulse Oximetry Sensor” filed Oct. 23, 2008, and of U.S. Provisional Patent Application Ser. No. 60/992,880 entitled “Noninvasive Photoplethysmographic Sensor Platform For Mobile Animals” filed Dec. 6, 2007

U.S. Pat. No. 7,857,768 and publication 2009-0149727 are incorporated herein by reference

### BACKGROUND INFORMATION

1. Field of the Invention

The present invention relates to an integrated appendage mounted, e.g., neck, pulse oximeter and blood pressure measurement apparatus for animal research.

2. Background Information

Pulse Oximetry

Pulse oximetry is a non invasive method that allows for the monitoring of the oxygenation of a subject's blood, generally a human or animal patient or an animal (or possibly human) research subject. The patient/research distinction is particularly important in animals because in research the data gathering is the primary focus, as opposed to care giving where it is the subject's well being, and as a result the physiologic data being obtained in the research of animals may, necessarily, be at extreme boundaries for the animal. Thus in animal research it is important to have medical devices capable of operating in physical parameters associated with the subject animal and which covers extreme values for such an animal.

As a brief history of pulse oximetry, it has been reported that in 1935 an inventor Matthes developed the first 2-wavelength earlobe O₂ saturation meter with red and green filters, later switched to red and infrared filters. This was the first device to measure O₂ saturation. Further in 1949 an inventor Wood added a pressure capsule to squeeze blood out of the earlobe to obtain zero setting in an effort to obtain absolute O₂ saturation value when blood was readmitted. The concept is similar to today's conventional pulse oximetry but suffered due to unstable photocells and light sources and the method was not used clinically. In 1964 an inventor Shaw assembled the first absolute reading ear oximeter by using eight wavelengths of light which was commercialized by Hewlett Packard, and its use was limited to pulmonary functions due to cost and size.

Effectively, modern pulse oximetry was developed in 1972, by Aoyagi at Nihon Kohden using the ratio of red to infrared light absorption of pulsating components at the measuring site, and this design was commercialized by BIOX/Ohmeda in 1981 and Nellcor, Inc. in 1983. Prior to the introduction of these commercial pulse oximeters, a patient's oxygenation was determined by a painful arterial blood gas, a single point measure which typically took a minimum of 20-30 minutes processing by a laboratory. It is worthy to note that in the absence of oxygenation, damage to the human brain starts in 5 minutes with brain death in a human beginning in another 10-15 minutes. Prior to its introduction, studies in anesthesia journals estimated US patient mortality as a consequence of undetected hypoxemia at 2,000 to 10,000 deaths per year, with no known estimate of patient morbidity. Pulse oximetry has become a standard of care for human patients since the mid to late 1980s. Pulse oximetry has been a critical research tool for obtaining associated physiologic parameters in humans and larger animals for at least as long.

In pulse oximetry a sensor is placed on a thin part of the subject's anatomy, such as a human fingertip or earlobe, or in the case of a neonate, across a foot, and two wavelengths of light, generally red and infrared wavelengths, are passed from one side to the other. Changing absorbance of each of the two wavelengths is measured, allowing determination of the absorbances due to the pulsing arterial alone, excluding venous blood, skin, bone, muscle, fat, etc. Based upon the ratio of changing absorbance of the red and infrared light caused by the difference in color between oxygen-bound (bright red) and oxygen unbound (dark red or blue, in severe cases) blood hemoglobin, a measure of oxygenation (the per cent of hemoglobin molecules bound with oxygen molecules) can be made.

The measured signals are also utilized to determine other physical parameters of the subjects, such as heart rate (pulse rate). Starr Life Sciences, Inc. has utilized pulse oximetry measurements to calculate other physiologic parameters such as breath rate, pulse distension, and breath distention, which can be particularly useful in various research applications.

Regarding human and animal pulse oximetry, the underlying theory of operation remains the same. However, consideration must be made for the particular subject or range of subjects in the design of the pulse oximeter, for example the sensor must fit the desired subject (e.g., a medical pulse oximeter for an adult human finger simply will not adequately fit onto a mouse finger or paw; and regarding signal processing the signal areas that are merely noise in a human application can represent signals of interest in animal applications due to the subject physiology). Consequently there can be significant design considerations in developing a pulse oximeter for small mammals or for neonates or for adult humans, but, again the underlying theory of operation remains substantially the same.

### Blood Pressure

Blood pressure refers to the force exerted by circulating blood on the walls of blood vessels, and constitutes one of the principal vital signs of a patient or subject (human or animal). The pressure of the circulating blood decreases as blood moves through arteries, arterioles, capillaries and veins; the term blood pressure generally refers to arterial blood pressure, i.e., the pressure in the larger arteries, arteries being the blood vessels which take blood away from the heart. Blood pressure in humans is most commonly measured via a device called a sphygmomanometer, which traditionally uses the height of a column of mercury to reflect the circulating pressure. Although many modern blood pressure devices no longer use mercury, blood pressure values are still universally reported in millimeters of mercury.

Systolic pressure is defined as the peak pressure in the arteries, which occurs near the beginning of the cardiac cycle; the diastolic pressure is the lowest pressure (at the resting phase of the cardiac cycle). The average pressure throughout the cardiac cycle is reported as mean arterial pressure; the pulse pressure reflects the difference between the maximum and minimum pressures means.

The ability to accurately and non invasively measure the systolic and diastolic blood pressure, in addition to other blood flow parameters in rodents, and other animals, is of great clinical value to the animal researcher. The general non-invasive blood pressure methodology for measuring blood pressure in rodents comprises utilizing a tail cuff placed proximally on the tail to occlude the blood flow. The subject's tail is threaded through the tail cuff. Upon deflation, one of several types of non invasive blood pressure sensors, placed distal to the occlusion cuff, will attempt to measure the blood pressure. There are several types of non invasive blood pressure sensor technologies: including photoplethysmography, piezoplethysmography, and volume pressure recording. Each of these methods will utilize an occlusion tail-cuff as part of the methodology.

It is worthwhile to note that direct blood pressure measurement in research applications is an invasive surgical procedure with the expense and time involved with invasive procedures, but this invasive procedure is often considered as a more precise measurement and this is used to compare the accuracy of non-invasive blood pressure technologies. Direct blood pressure should be performed on the rodent's carotid artery, rather than the femoral artery.

Photoplethysmography based blood pressure measurements in rodents is the first and oldest sensor type and is a light-based technology. photoplethysmography (PPG) is described above in general. The aim for PPG blood pressure measurements is to record the first appearance of the pulse when it re-enters the tail artery during the deflation cycle of the proximal occlusion cuff. Photoplethysmography blood pressure measurement utilizes a standard light source or a LED light source to record the pulse signal wave. As such, this light-based plethysmographic method uses the light source to illuminate a small spot on the tail and attempts to record the pulse.

A second non invasive blood pressure sensor technology is piezoplethysmography. Piezoplethysmography and photoplethysmography both require the same first appearance of pulse in the tail to record the systolic blood pressure and heart rate. Whereas photoplethysmography uses a light source to record the pulse signal, piezoplethysmography utilizes piezoelectric ceramic crystals to record blood pressure readings. From a technical point of view, piezoplethysmography acquires blood pressure readings when the re-appearance of the pulse in the rodent's tail produces a change that can be equated to a voltage shift. The voltage shift momentarily deforms the ceramic crystals and the change is converted to millimeters of mercury for blood pressure readings.

A third sensor technology is volume pressure recording that utilizes a differential pressure transducer to non-invasively measure the blood volume in the tail of a subject.

Representative, commercial rodent tail cuff blood pressure monitoring devices are available from IITC, Life Science, Inc.; Columbus Instruments, Inc.; and Kent Scientific.

Non-invasive tail mounted blood pressure measurement systems for animals should be designed to comfortably warm the animal, reduce the animal's stress and enhance blood flow to the tail. The rodent's core body temperature is very important for accurate and consistent blood pressure measurements. The animal must have adequate blood flow in the tail to acquire a blood pressure signal. Thermo-regulation is the method by which the animal reduces its core body temperature, dissipates heat through its tail and generates tail blood flow. Anesthetized animals may have a lower body temperature than awake animals so additional care must be administered to maintain the animal's proper core body temperature.

An infrared warming blanket or a re-circulating water pump with a warm water blanket are conventional methods to maintain the animal's proper core body temperature. The animal should preferably be warm and comfortable but never hot. Extreme care must be exercised to never overheat the animal. Hot air heating chambers, heat lamps, heating platforms that apply direct heat to the animal's feet have been suggested as well as tail cuff heating devices. However care must be taken with any thermal regulation system to avoid overheating the animal that may increase the animal's respiratory rate, thereby increasing the animal's stress level. These conditions can elicit poor thermo-regulatory responses and may create inconsistent and inaccurate blood pressure readings.

The above discussion notes that blood pressure monitoring in small mammals is somewhat well developed and a very useful tool for researchers. The tail based measurements still provides unique problems for measuring physiologic measurements in rodents. Further, pulse oximetry has been expanded to be effectively applied to small mammals, such as mice as shown in the MouseOx® brand small mammal pulse oximeter available from the assignee, and has provided further useful tools to researchers. There remains a need in the art to effectively expand the useful tools applicable to researchers, to simplify there use and improve the physiologic results.

### SUMMARY OF THE INVENTION

One embodiment of the present invention provides an integrated non-invasive blood pressure monitor and pulse oximeter system that includes a blood flow occlusion member configured to be secured to a subject's tail or other appendage and configured to selectively occlude blood flow through the tail or other appendage; a sensor coupled to the blood flow occlusion member configured to detect a degree of operation of the blood flow occlusion member; light sources configured to be coupled to the tail or other appendage and configured to selectively direct light of at least two different wavelengths into the appendage; at least one light receiver configured to be coupled to the tail or other appendage and configured to selectively receive a signal associated with light that has been directed into the tail or other appendage from the light sources; and a controller coupled to the blood flow occlusion member for controlling the blood flow occlusion member, and coupled to the sensor and the at least one light receiver for receiving data there from, wherein the controller is configured to selectively determine blood pressure parameters from the data and pulse oximeter parameters from the data.

Within the meaning of this application occlusion of the blood flow means restriction of the blood flow. Occlusion of the blood flow includes partial occlusion and full or total occlusion. Full or total blood flow occlusion within the meaning of this application means completely blocking the blood flow, while partial occlusion means a restriction of a measurable portion of the blood flow less than full or total occlusion. The term appendage within this application means the non-torso portion of the subject, including the tail, the head and neck, and each limb.

One aspect of the present invention provides a tail mounted blood pressure monitor comprising an animal holder containing an animal; a tail blood flow occlusion member coupled to the holder and configured to be secured to a subject animal's tail and configured to selectively occlude blood flow through the tail, wherein the tail blood flow occlusion member includes two housing halves that are selectively movable toward and away from each other; a sensor coupled to the tail blood flow occlusion member configured to detect a degree of operation of the tail blood flow occlusion member; at least one light source configured to be coupled to the tail in a position closer to the distal end of the tail than the position of the tail blood flow occlusion member, and configured to selectively direct light into the tail; at least one light receiver configured to be coupled to the tail in a position closer to the distal end of the tail than the position of the tail blood flow occlusion member, and configured to selectively receive a signal associated with light that has been directed into the tail from the at least one light source; and a controller coupled to the tail blood flow occlusion member for controlling the tail blood flow occlusion member, and coupled to the sensor and the at least one light receiver for receiving data there from.

One embodiment of the present invention provides an integrated neck mounted non-invasive blood pressure monitor and pulse oximeter system that includes a blood flow occlusion member configured to be secured to a subject's neck and configured to selectively occlude blood flow through the neck; a sensor coupled to the blood flow occlusion member configured to detect a degree of operation of the blood flow occlusion member; light sources configured to be coupled to the neck and configured to selectively direct light of at least two different wavelengths into the neck; at least one light receiver configured to be coupled to the neck and configured to selectively receive a signal associated with light that has been directed into the neck from the light sources; and a controller coupled to the blood flow occlusion member for controlling the blood flow occlusion member, and coupled to the sensor and the at least one light receiver for receiving data there from, wherein the controller is configured to selectively determine blood pressure parameters from the data and pulse oximeter parameters from the data.

These and other advantages of the present invention will be clarified in the brief description of the preferred embodiment taken together with the drawings in which like reference numerals represent like elements throughout.

### BRIEF DESCRIPTION OF THE DRAWINGS

FIG. 1 is a schematic top plan view of an integrated blood pressure monitor and pulse oximeter according to one aspect of the present invention;

FIG. 2 is a schematic front view of a blood flow occlusion member in accordance with one aspect of the present invention;

FIG. 3 is a schematic front view of a blood flow occlusion member in accordance with another aspect of the present invention;

FIG. 4 is a schematic top plan view of an integrated blood pressure monitor and pulse oximeter according to one aspect of the present invention;

FIG. 5 is a schematic front view of a blood flow occlusion member in accordance with another aspect of the present invention;

FIG. 6 is a schematic front view of a blood flow occlusion member in accordance with another aspect of the present invention;

FIG. 7 is a schematic front view of a blood flow occlusion member in accordance with another aspect of the present invention;

FIG. 8 is a schematic front view of a mounting clip with associated light sources and receivers in accordance with one aspect of the present invention;

FIG. 9 is a schematic view of an integrated blood pressure monitor and pulse oximeter according to one aspect of the present invention; and

FIG. 10 is a schematric view of an integrated blood pressure monitor and pulse oximeter according to one aspect of the present invention.

### DESCRIPTION OF THE PREFERRED EMBODIMENTS

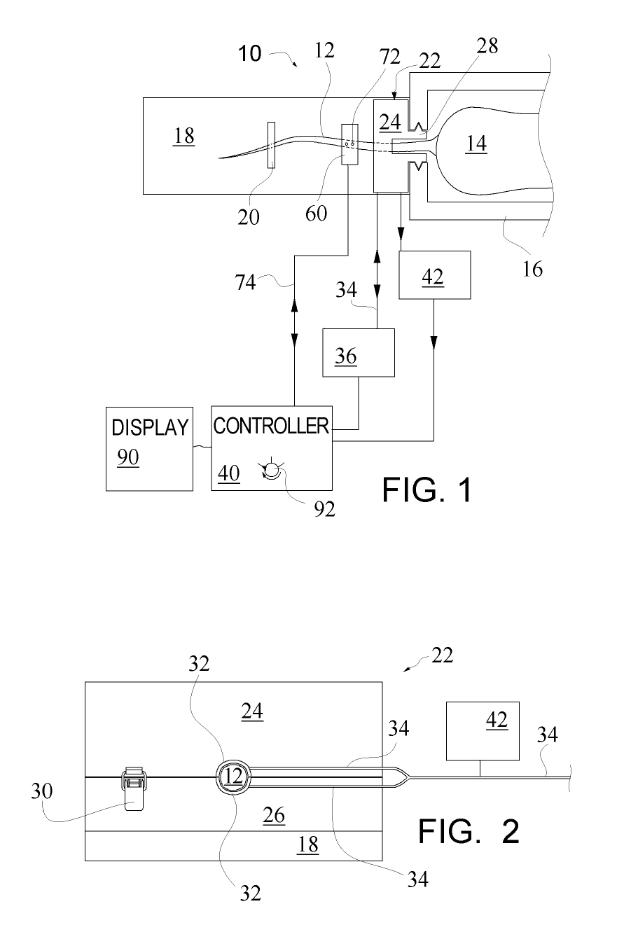

FIG. 1 is a schematic view of integrated tail mounted blood pressure monitor and pulse oximeter system 10 in accordance with the present invention. The system is designed for the tail 12 of a mouse 14, but can be used with any small mammal.

The mouse 14 is held within an animal holder 16, also known as an animal restraint tube. Animal restraint tubes most often used in research are constructed generally of a clear plastic and have a slit that runs the entire length along the top of the tube. The tube is open on one end, and is closed on the other end (and only the closed end is shown in FIGS. 1 and 4), but the slit described above is joined on the closed end by a slit that runs to the center of the end cap. To use the tube, a researcher grabs the animal's tail 12, and pulls it through the slit from the open end of the tube, toward the closed end. Once the animal, or mouse 14, is pulled all of the way into the tube, a restricting ring or plate is slid into the open end of the tube to allow the user to push the animal, or mouse 12, into the tube and restrict its motion. With the securing of the restricting ring the animal, or mouse 12, is effectively immobilized and the research can proceed. The holder 16 used with the system 10 of the present invention includes a tail support board 18 as part of the holder 12 and the board 18 extends beyond the tail opening and includes slits or the like for one or more tail tie down members 20 that can secure the tail 12 to the support board 18.

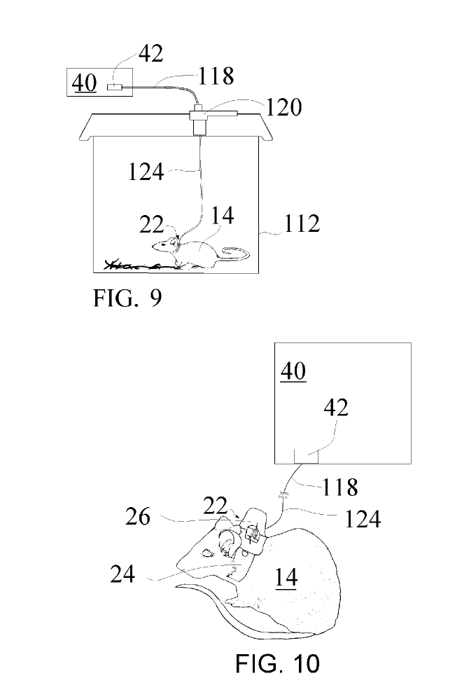

The system 10 according to the present invention includes a tail blood flow occlusion member 22 configured to be secured to an animal subject's tail 12 and selectively occlude blood flow through the tail 12. As noted above, occlusion of the blood flow means restriction of the blood flow. Here on the tail 12 the occlusion member is configured to operate in both partial occlusion and full or total occlusion. Full or total blood flow occlusion within the meaning of this application means completely blocking the blood flow and allows the blood pressure sensors to operate in generally a conventional fashion, while partial occlusion means a restriction of a measurable portion of the blood flow less than full or total occlusion, and will utilize the parameters as discussed further in connection with FIGS. 9-10, below. Specifically, in partial occlusion modes distension measurements of the pulse oximeter at, at least, two distinct levels of operation of the occlusion member 22 are used as a blood pressure parameter for the animal.

FIG. 2 illustrates a first embodiment of the tail blood flow occlusion member 22 using a two housing halves 24 and 26 that are selectively movable toward and away from each other. The lower half 26 can be secured to the tail support board 18 and the upper half 24 can be moveable in a slide 28 that engages rails formed in the holder 16. The weight of the upper half 24 may be such that it is held in a closed position via gravity, or a latch 30 may be used to secure the halves 24 and 26 together in the closed, operative position. With the formation of the tail blood flow occlusion member as two halves the tail 12 need not be “threaded” through a closed opening. Once the tail 12 is properly positioned on the board 18 on top of the lower half 26, the upper half 24 can be slid into position.

The upper and lower halves 24 and 26 include aligned tail receiving recesses as shown. Further each recess includes a respective inflatable tail cuff portion 32. With the tail 12 in the recesses and the upper and lower halves positioned together, the inflatable tail cuff portions substantially encircle the tail 12. Inflation/deflation lines 34 extend to each tail portion 32 for selectively inflating and deflating the tail cuff portions 32 from an actuator 36, such as a pump, controlled via controller 40. A sensor 42 is coupled to the tail cuff portions 32 in a manner to determine the relative pressure within the cuff portions 32 whereby the sensor 42 is configured to detect a degree of operation of the tail blood flow occlusion member 22. The sensor 42 is coupled to the controller 40 to supply data thereto. In addition to conventional operation as a cuff sensor in a blood pressure device, the sensor 42 can be used to indicate when the tail blood flow occlusion member 22 is not in use and the pulse oximetry measurements can be made with the system 10 without significant problems, assuming there is blood flow in the tail or other appendage being measured.

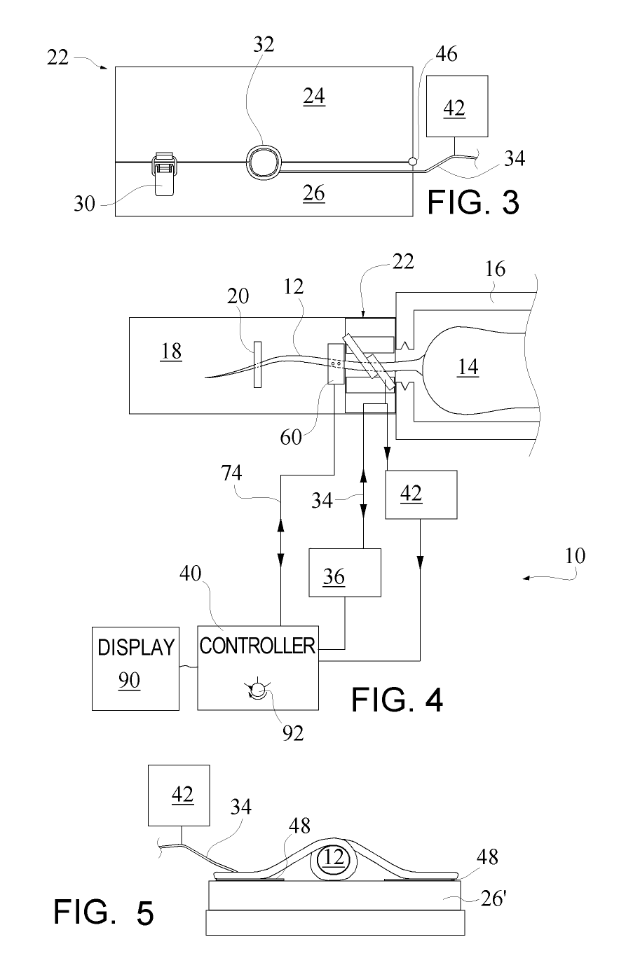

FIG. 3 illustrates a second embodiment of the tail blood flow occlusion member 22 using a two housing halves 24 and 26 that are selectively movable toward and away from each other. In this embodiment the halves 24 and 26 are pivoted together at pivot 46. A latch 30 may be used to secure the halves 24 and 26 together in the closed, operative position. This embodiment may be easily positioned “vertically” whereby the parting line between the halves is vertical so that it opens upwardly to assist in the tail placement. The attachment of one half 24 or 26 to the board 18 can be made to accommodate the open position of the other half for easy placement of the tail 12. With the formation of the tail blood flow occlusion member as two halves the tail 12 need not be “threaded” through a closed opening. Once the tail 12 is properly positioned within the opened halves 24 and 26, the halves 24 and 26 are closed and latched.

The halves 24 and 26 include aligned tail receiving recesses as shown. Further the recesses include a single inflatable tail cuff portion 32. With the tail 12 in the recesses and the halves 24 and 26 positioned together, the inflatable tail cuff portion 32 substantially encircles the tail 12. An inflation/deflation line 34 extends to the tail portion 32 for selectively inflating and deflating the tail cuff portion 32 from an actuator or pump 36 controlled via controller 40. A sensor 42 is coupled to the tail cuff portion 32 in a manner to determine the relative pressure within the cuff portion 32, whereby the sensor 42 is configured to detect a degree of operation of the tail blood flow occlusion member 22. The sensor 42 is coupled to the controller 40 to supply data thereto. In addition to conventional operation as a cuff sensor in a blood pressure device, the sensor 42 can be used to indicate when the tail blood flow occlusion member 22 is not in use and the pulse oximetry measurements can be efficiently made with the system 10.

FIGS. 4 and 5 illustrate a further inflatable cuff 32 version of the blood flow occlusion member 22 in accordance with the present invention. In this embodiment the inflatable cuff 32 is wrapped around the tail 12 and secured at the ends thereof to a base 26′ that is secured to the board 18. Releasable fasteners, such as hook and loop type fasteners 48 can be utilized to secure the ends of the cuff 32 to the base 26′. The material forming the cuff 32 can engage with the fastener material 48 or additional material that does engage with the material 48 can be added to the ends of the cuff 32 as needed. Further, the base 26′ can be eliminated and the fasteners 48 secured directly to the board 18.

With the formation of the tail blood flow occlusion member 22 with a wrap around tail cuff 32, the tail 12 need not be “threaded” through a closed opening. Once the tail 12 is properly positioned on the un-wrapped (i.e. laid open) cuff 32, the ends of the cuff are wrapped around the tail 12 and secured to the base 26′, whereby the inflatable tail cuff portion 32 substantially encircles the tail 12. An inflation/deflation line 34 extends to the tail cuff portion 32 for selectively inflating and deflating the tail cuff portion 32 from an actuator or pump 36 controlled via controller 40. A sensor 42, as in the embodiments described above, is coupled to the tail cuff portion 32 in a manner to determine the relative pressure within the cuff portion 32, whereby the sensor 42 is configured to detect a degree of operation of the tail blood flow occlusion member 22. The sensor 42 is coupled to the controller 40 to supply data thereto. Again, with this embodiment, in addition to conventional operation as a cuff sensor in a blood pressure device, the sensor 42 can be used to indicate when the tail blood flow occlusion member 22 is not in use and the pulse oximetry measurements can be efficiently made with the system 10, assuming there is blood flow in the tail or other appendage of the subject.

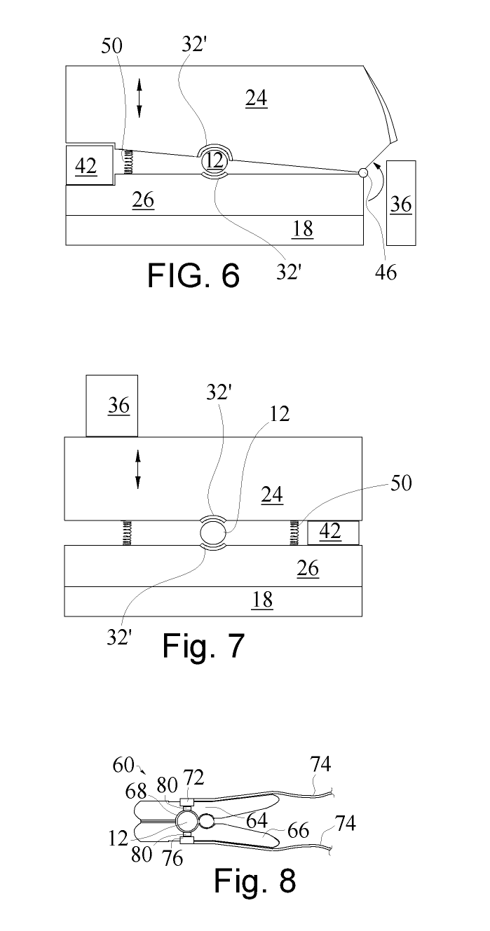

FIG. 6 illustrates a further embodiment of the tail blood flow occlusion member 22 using a two housing halves 24 and 26 that are selectively movable toward and away from each other, without using an inflatable cuff. In this embodiment the halves 24 and 26 are pivoted together at pivot 46. The halves 24 and 26 include aligned tail receiving recesses with each recess including a tail engaging member 32′. The tail engaging member 32′ may be a rubber strip or other resilient member to distribute the force of the closing halves 24 and 26. Unlike earlier versions the recesses in the halves 24 and 26 do not completely accommodate the tail 12 as it is the movement of the halves 24 and 26 together that acts to occlude the blood flow. The recesses could be eliminated completely from the halves 24 and 26, but the presence of some recess portion is believed to assist in tail placement. An actuator 36, such as a linear motor or solenoid, controlled via controller 40 is used to move the halves 24 and 26 in a controlled manner toward and away from each other.

A sensor 42 is coupled to halves 24 and 26 and/or to the actuator 36 in a manner to determine the relative position or force on the tail 12, whereby the sensor 42 is configured to detect a degree of operation of the tail blood flow occlusion member 22. The sensor 42 may be a position sensor or a force sensor. In this embodiment the data from the sensor 42 must be calibrated to equate to an associated pressure on the tail 12 for the blood pressure calculations. However there is believed to be a correlation to the position of the halves 24 and 26, or the force on the sensor 42 and the associated pressure applied to the tail 12. The sensor 42 is coupled to the controller 40 to supply data thereto. In addition to conventional operation as a cuff sensor in a blood pressure device, the sensor 42 can be used to indicate when the tail blood flow occlusion member 22 is not in use and the pulse oximetry measurements can be efficiently made with the system 10. Again, with the formation of the tail blood flow occlusion member 22 as two halves 24 and 26 the tail 12 need not be “threaded” through a closed opening.

FIG. 7 illustrates a further embodiment of the tail blood flow occlusion member 22 using a two housing halves 24 and 26 that are selectively movable toward and away from each other, without using an inflatable cuff. The embodiment of FIG. 7 is similar to the embodiment of FIG. 6 in that the halves 24 and 26 do not completely accommodate the tail 12 and it is the movement of the halves 24 and 26 together that acts to occlude the blood flow through the tail 12. The halves 24 and 26 include aligned tail receiving recesses with each recess including a tail engaging member 32′. The tail engaging member 32′ may be a rubber strip or other resilient member to distribute the force of the closing halves 24 and 26. Again, the recesses could be eliminated completely from the halves 24 and 26, but the presence of some recess portion is believed to assist in tail placement. An actuator 36, such as a linear motor or solenoid, controlled via controller 40 is used to move the halves 24 and 26 in a controlled manner toward and away from each other. A sensor 42 is coupled to halves 24 and 26 and/or to the actuator 36 in a manner to determine the relative position or force on the tail 12, whereby the sensor 42 is configured to detect a degree of operation of the tail blood flow occlusion member 22. The sensor 42 may be a position sensor or a force sensor. In this embodiment the data from the sensor 42 must be calibrated to equate to an associated pressure on the tail 12 for the blood pressure calculations. The sensor 42 is coupled to the controller 40 to supply data thereto. In addition to conventional operation as a cuff sensor in a blood pressure device, the sensor 42 can be used to indicate when the tail blood flow occlusion member 22 is not in use and the pulse oximetry measurements can be efficiently made with the system 10. Again, with the formation of the tail blood flow occlusion member 22 as two halves 24 and 26 the tail 12 need not be “threaded” through a closed opening. The difference between embodiments 6 and 7 is that the motion of the halves 24 and 26 in FIG. 7 is a linear motion similar to the embodiment of FIGS. 1 and 2.

The embodiments of FIGS. 6 and 7 may further include springs 50 for biasing the halves to an open position that does not place pressure on the tail that could otherwise effect pulse oximetry measurements. Further, with an actuator 36 the sensor 42 may be incorporated into the actuator 36, such as a motor encoder or the like. Further, the actuator could possibly be the addition of given weights, such as pumping water into a receiving reservoir on the upper surface of the upper halve 26, whereby the “sensor” 42 is merely a measurement of the amount of weight that has been added, wherein the weight total will correlate to a specific pressure on the tail. Many alternative embodiments for the blood flow occlusion member 22 are possible within the scope of the present invention and these examples are merely illustrative of some of these possibilities. All of these embodiments provide easy tail placement over earlier tail cuff designs.

FIG. 8 illustrates a tail mounting clip 60 for securely mounting physiologic light transmitting and receiving sensors onto the tail 12 in accordance with one embodiment of the present invention. The clip 60 of the present invention provides spring biased halves 64 and 66 pivoted together and biased to a closed position. A transverse circular groove 68 on the tail engaging faces of the halves 64 and 66 is configured to receive and locate the tail 12 therein. The one halve 64 includes a plurality of light sources 72, with leads 74 extending to controller 40. The other halve 66 includes at least one receiver 76, or photo-detectors, configured to receive the light transmitted through the tail 12 from the light sources 72 and coupled to the controller 40 via leads 74. The light sources supply light of at least two distinct wavelengths, generally red and infrared, as is conventional in pulse oximetry. The use of the clips, in general, is known in the pulse oximetry art for coupling associated sensors, such as the LED sources and photo-detectors. The groove 68 makes the clip 60 particularly well suited as a tail mounting element. The groove 68 may be circular in cross section and extends generally perpendicular across the halves 64 and 66 and is preferably sized to accommodate a conventional subject's tail 12 as shown schematically in FIG. 8. The clip 68 may be formed of a non-translucent plastic material and designed to minimize ambient light received by the receivers 76 whereby apertures 80 are supplied to accommodate the desired light transmission and receipt.

In the illustrated but non-limiting embodiment of the present invention the clip 60 is a spring-loaded pivoted body type clamp. The halves 64 and 66 could be attached with some other method, including adhesives, magnetic elements, tape, or combinations thereof without departing from the scope of the present invention. The illustrated embodiment also possesses the rounded, transverse groove 48 on both halves 64 and 66 of the clip 60, but a single tail receiving groove could be provided on only one clip half. Additionally, the groove 48 could have a variable cross-sectional shape, and does not have to be limited to semi-circular. It could also be V-groove, or square in cross-section. The illustrated embodiment uses groove 48 running transverse to the direction of the clip 60, it could also run axially with the clip 60, or at any angle between.

As shown the light sources 72 are configured to be coupled to the tail 12 in a position closer to the distal end of the tail 60 than the position of the tail blood flow occlusion member 22, and configured to selectively direct light of at least two different wavelengths into the tail 12. Further the at least one light receiver 76 is configured to be coupled to the tail 12 in a position closer to the distal end of the tail 12 than the position of the tail blood flow occlusion member 22, and is configured to selectively receive a signal associated with light that has been directed into the tail 12 from the light sources 72.

The controller 40 coupled to the tail blood flow occlusion member 22 for controlling the tail blood flow occlusion member 22, and is coupled to the sensor 42 and the light receivers 76 for receiving data there from.

The key aspect of the present invention is that the controller 40 is configured to selectively determine blood pressure parameters from the data and pulse oximeter parameters from the data. In one operational mode the blood flow occlusion member 22 and clip 60, with controller 40 combine to form a photoplethysmography based blood pressure measurement device. As noted above the aim of such a device, when in the blood pressure device mode and operating in full occlusion mode, is to record the first appearance of the pulse when it re-enters the tail artery during the deflation cycle of the proximal occlusion cuff. Conventional photoplethysmography blood pressure measurement utilizes a standard light source, or a LED light source, to record the pulse signal wave. The signal processing required for such determinations is known to those of ordinary skill in this art, and representative example of such processing is found in the MouseOx® brand small animal pulse oximeters available from the assignee since late 2005 and to the present filing of this application. The results of such calculations can be displayed on an associated display 90. In the blood pressure monitoring mode it is common to have the device cycle through measurements periodically.

In a second operational mode the system 10 obtains pulse oximeter measurements from clip 60. The signal processing of such devices is known from Starr Life Sciences Mouse Ox® brand pulse oximeters, as noted above, and such results can be displayed to the display 90. The sensor 42 can be used in the pulse oximetry mode to assure that the blood flow occlusion member is not significantly obstructing blood flow through the tail 12, which could other-wise detrimentally affect the results of the pulse oximetry measurements. A selector 94 can be provided on the controller to allow the user to select between pulse oximetry measurements with the system 10, blood pressure measurements with the system 10, or both. When selecting both it is expected that the system 10 will cycle through the blood pressure measurements on a given timing cycle (e.g. one blood pressure measurement every 3 minutes) and obtain pulse oximetry measurements during the “off” cycles.

An alternative system 10 is shown in FIG. 9 which includes a neck blood flow occlusion member 22 configured to be secured to an animal subject's neck and selectively partially occlude blood flow through the neck. As noted above, occlusion of the blood flow means restriction of the blood flow. Here on the neck the occlusion member is configured to operate in generally only in partial occlusion meaning a restriction of a measurable portion of the blood flow less than full or total occlusion. Distension measurements of the pulse oximeter at, at least, two distinct levels of operation of the occlusion member 22 are used as a blood pressure parameter for the animal.

The neck blood flow occlusion member 22 uses two housing halves 24 and 26 (shown in FIG. 10) that are selectively movable via a pivot point toward and away from each other. The controller 40 coupled to the neck blood flow occlusion member 22 for controlling the tail blood flow occlusion member 22, and is coupled to the sensor 42 and the light receivers for receiving data there from. The controller 40 is shown schematically in FIG. 9 and can be formed as a component of a laptop or desktop computer. A conventional controller cable 118 extends from the controller 40 for transmitting control and power signals from the controller and data back to the controller 40. The controller cable 118 is coupled to a rotation coupling 120, also called a swivel link. A neck clip cable 124 is attached to and extends from the rotation coupling 120. The rotation coupling 120 allows relative rotation between the controller cable 118 and the collar cable 124. The rotation coupling 120 provides a convenient location for mounting to the confinement unit 112. The use of the swivel link or rotation coupling 120 allows the animal, e.g. mouse 14, to be effectively freely roaming within the area of the unit 112, wherein twisting of the cables is avoided. The swivel link or rotation coupling 120 also serves to effectively divide the system 10 into an animal specific portion and the controller portion, whereby the controller 40 can be easily used with a large number of animal specific portions in a serial fashion. Further, it allows for easy replacement of the animal portion which is anticipated to have a shorter life span than the controller 40. It should be clear that the embodiment of FIG. 10 allows for use of the system 10 with non-anesthetized animals.

The present invention does anticipate that the controller 40 may be simultaneously (e.g. a parallel attachment) connected to a number of animal specific portions through separate cables 118 to allow for obtaining numerous study results at the same time, but this configuration does not eliminate the advantages of the coupling 120.

The neck of small mammals such as rats and mice allows for a number of advantages for photoplethysmographic pulse oximetry measurements. The necks of animals of the sub-order muroidia tend to allow for both transmittance and reflective pulse oximetry measurements. Transmittance pulse oximetry is where the received light is light that has been transmitted through the perfuse tissue, whereas in reflective pulse oximetry the representative signal is obtained from light reflected back from the perfuse tissue. Each technique has its unique advantages. Transmittance techniques often result in a larger signal of interest, which is very helpful in small animals that have very small quantities of blood being measured to begin with. Reflective techniques can be used in environments that do not allow for transmittance procedures (e.g. the forehead of a human).

Further, the neck region of the animal offers an area with a relatively large blood flow for the animal, which will improve the accuracy of the measurements. In addition to increased blood flow, the blood flow is present under substantially all conditions. In other areas of the animal, such as the legs, paws and tail, the animal will often cut off blood flow under a variety of conditions. For example if the animal is cold or sufficiently agitated the blood flow to the tail can be shunted. The neck, in contrast represents an area of the animal that will always maintain a constant blood flow for measurements. The brain is the last organ to have blood flow reduced in response to some sort of physiologic challenge, such as cold, stress or blood loss, which can cause a shock response. In the case of shock, blood flow is reduced to the extremities, but is still always supplied to the brain, and that blood must necessarily pass through the neck. Additionally, because of the continuous flow of blood through the neck to the brain, it is not necessary to heat the animal to aid perfusion, as can be the case for measurements on the tail or the extremities.

The neck clip, shown in greater detail in FIG. 10 also provides a bite proof location for the sensor mounting. In attempting to remove the sensors the biting of most animals, particularly animals of the sub-order muroidia, will be stronger than the clawing, and the neck location prevents the biting attacks as the animal cannot reach the neck mounted clip.

A further advantage of this neck clip design is a simple one handed application to the neck of an animal by only a single user. The clip can be designed to be light and unobtrusive, thus it also can be used to make measurements on conscious animals that are free-roaming within the boundaries of the attached wire 124.

The clip is designed to have two halves that are connected with a pivot pin at the top pivot point or hinge. A torsion spring may be positioned by passing the pin through it so that it resides between the clip halves and can leverage off of both clamp handles. However the use of The LED(s) and photodiode(s) forming the sensors can reside opposite each other on the inside of the clip with wires 124 protrude through holes in the handles of the clip and pass to a coupling 120.

The controller 40 also operates the occlusion member 22, which in the neck mounted version can simply be actuators more securely clamping the halves of the neck clip onto the animal to measurably restrict, but generally not to cut off, blood flow. The sensor 42 is coupled to the controller to measure the relative amount of force or pressure used on the occlusion member 22.

In the neck mounted embodiment, the system uses distension measurements of the pulse oximeter at distinct occlusion points as blood pressure parameters for the animal. Specifically the system 10 can utilize the ratio of calculated distention measurements, preferably pulse distention, to the force or degree of operation of the occlusion member at a series of distinct occlusion points. Another way of explaining the operation is that the calculated distension measurements at each of a series of degrees of operation of the occlusion member will graphically demonstrate a curve that is indicative of the blood pressure of the animal. The slope, inflection points, curvature, asymptotes, maximum, etc of this graphical relationship can all be used as blood pressure parameters of the animal. Different parameters will have different applicability to the researchers. The present invention contemplates that the blood pressure parameters for partial blood flow occlusion mode are based upon the distension measurements of the pulse oximeter taken at distinct operational points of the occlusion member.

To reiterate, the blood pressure system for the neck mounted clip operates as follows. Measurements are taken of the distension measurements (pulse, breath and the combination) of the animal at a first point, namely no occlusion, or full flow. Here the clip is attached but not measurably decreasing blood flow. The occluding member or clip is moved to a second stage, namely tighter (without completely occluding flow) and additional measurements are taken of the distension measurements (pulse, breath and the combination) of the animal at this second point. Preferably the occluding member or clip is moved to a third stage, namely tighter (without completely occluding flow) and additional measurements are taken of the distension measurements (pulse, breath and the combination) of the animal at this third point. The process can be repeated to obtain a series of distension measurements that are related to distinct and measurable (as measured by sensor 42) occlusion points. The distension measurements relative to the occlusion member points will provide for blood pressure parameters for the animal.

It is believed the system 10 can be calibrated to provide direct blood pressure measurements in conventional units. Further it is anticipated that the system will cycle through blood pressure measurements periodically as desired by the researcher. Further, although the neck clip embodiment is anticipated to operate only in partial occlusion mode to obtain blood pressure parameters, the tail, or other appendage clip can effectively operate in partial occlusion and full occlusion mode, or both.

Although the present invention has been described with particularity herein, the scope of the present invention is not limited to the specific embodiment disclosed. It will be apparent to those of ordinary skill in the art that various modifications may be made to the present invention without departing from the spirit and scope thereof. For example, although particularly well suited for the tail of a subject animal, the present invention can be deployed on other appendages of a subject animal.

What is claimed is:

1. An integrated blood pressure monitor and pulse oximeter system comprising:
A blood flow occlusion member configured to be secured to a subject and selectively occlude blood flow through the tail;
A sensor coupled to the blood flow occlusion member configured to detect a degree of operation of the blood flow occlusion member;
Light sources configured to be coupled to the animal and configured to selectively direct light of at least two different wavelengths into the animal;
At least one Light receiver configured to be coupled to the animal and configured to selectively receive a signal associated with light that has been directed into the animal from the light sources;
Controller coupled to the blood flow occlusion member for controlling the blood flow occlusion member, and coupled to the sensor and the light receivers for receiving data there from, wherein the controller is configured to selectively determine blood pressure parameters from at least distension parameters.

2. The integrated blood pressure monitor and pulse oximeter system according to claim 1 wherein the distension measurements include both pulse distension, and breath distention.

3. The integrated blood pressure monitor and pulse oximeter system according to claim 1 wherein the blood flow occlusion member includes at least one inflatable cuff portion.

4. The integrated blood pressure monitor and pulse oximeter system according to claim 1 wherein the blood flow occlusion member includes two housing halves that are selectively movable toward and away from each other.

5. The integrated blood pressure monitor and pulse oximeter system according to claim 4 wherein the two housing halves are configured to be attached to the neck of the animal.

6. The integrated tail mounted blood pressure monitor and pulse oximeter system according to claim 4 wherein the movement of the housing halves toward each other is configures to selectively restrict blood flow.

7. The integrated blood pressure monitor and pulse oximeter system according to claim 1 wherein the blood flow occlusion member includes an inflatable cuff portion that is wrapped around the animal.

8. The integrated blood pressure monitor and pulse oximeter system according to claim 1 further including an animal holder containing the animal and wherein the tail blood flow occlusion member is secured to the holder.

9. A blood pressure monitor comprising:
A neck mounted blood flow occlusion member configured to be secured to a subject animal's neck and selectively partially occlude blood flow through the neck, wherein the neck blood flow occlusion member includes two housing halves that are selectively movable toward and away from each other;
A sensor coupled to the neck blood flow occlusion member configured to detect a degree of operation of the neck blood flow occlusion member;
At least one light source configured to be coupled to the neck configured to selectively direct light into the neck;
At least one Light receiver configured to be coupled to the neck and configured to selectively receive a signal associated with light that has been directed into the neck from the at least one light source; and
Controller coupled to the neck blood flow occlusion member for controlling the neck blood flow occlusion member, and coupled to the sensor and the at least one light receiver for receiving data there from.

10. The neck mounted blood pressure monitor according to claim 9 wherein the neck blood flow occlusion member includes pivotable clip halves.

11. The neck mounted blood pressure monitor according to claim 9 wherein the movement of the housing halves toward each other is configures to selectively restrict blood flow through the neck

12. The neck mounted blood pressure monitor according to claim 9 wherein the light source is configured to selectively direct light of at least two different wavelengths into the neck, and wherein the controller is configured to selectively determine blood pressure parameters from the data and pulse oximeter parameters from the data, and wherein the pulse oximeter parameters include breath rate.

13. The tail mounted blood pressure monitor according to claim 9 wherein the light source is configured to selectively direct light of at least two different wavelengths into the tail, and wherein the controller is configured to selectively determine blood pressure parameters from the data and pulse oximeter parameters from the data, and wherein the pulse oximeter parameters include pulse distension.

14. The tail mounted blood pressure monitor according to claim 9 wherein the controller utilizes distension measurements to calculate blood pressure parameters.

15. An integrated blood pressure monitor and pulse oximeter system comprising:
A blood flow occlusion member configured to be secured to a subject's appendage and selectively occlude blood flow through the appendage;
A sensor coupled to the blood flow occlusion member configured to detect a degree of operation of the blood flow occlusion member;
A mounting clip attachable to the appendage in a position closer to the distal end of the appendage than the position of the blood flow occlusion member;
Light sources carried on the mounting clip and configured to direct light of at least two different wavelengths into the appendage;
At least one Light receiver carried on the mounting clip and configured to selectively receive a signal associated with light that has been directed into the appendage from the light sources;
Controller coupled to the blood flow occlusion member for controlling the blood flow occlusion member, and coupled to the sensor and the light receivers for receiving data there from, wherein the controller is configured to selectively determine blood pressure parameters from the data and pulse oximeter parameters from the data.

16. The integrated blood pressure monitor and pulse oximeter system according to claim 15 wherein the pulse oximeter parameters include breath rate, pulse distension, and breath distention.

17. The integrated blood pressure monitor and pulse oximeter system according to claim 15 wherein the blood flow occlusion member includes two housing halves that are selectively movable toward and away from each other and configured to be attached to the neck of the animal.

18. The integrated tail mounted blood pressure monitor and pulse system oximeter according to claim 17 wherein a spring bias holds the two housing halves in a relaxed, non-blood flow occluding position.

19. The integrated blood pressure monitor and pulse oximeter system according to claim 17 wherein the movement of the housing halves toward each other is configures to selectively cut off blood flow through the appendage.

20. The integrated blood pressure monitor and pulse oximeter system according to claim 15 wherein the blood flow occlusion member includes an inflatable cuff portion that is wrapped around the appendage, and further including a subject holder containing the subject and wherein the blood flow occlusion member is secured to the holder.
